(19) United States
(12) Patent Application Publication
(10) Pub. No.: US 2022/0354646 A1
(43) Pub. Date: Nov. 10, 2022

(54) ANCHOR DESIGNS CONFIGURED FOR ANCHOR MIGRATION/BACKOUT CONTROL

(71) Applicant: Boston Scientific Scimed, Inc., Maple Grove, MN (US)

(72) Inventors: Gregory Y. Lee, Eden Prairie, MN (US); Michelle C. Boucha-Rayle, Minnetonka, MN (US); James M. Anderson, Corcoran, MN (US); Joshua M. Inouye, Brooklyn Park, MN (US); John A. Zanussi, Minneapolis, MN (US); Jeffrey Schneider, Champlin, MN (US); Christopher Korenczuk, New Brighton, MN (US); Kenny Dwight Bruner, Windsor, CA (US); Graham Krumpelmann, Stillwater, MN (US)

(21) Appl. No.: 17/624,468

(22) PCT Filed: Aug. 3, 2020
(86) PCT No.: PCT/US20/44740

§ 371 Date: Jan. 3, 2022

Related U.S. Application Data
(60) Provisional application No. 62/882,027, filed on Aug. 2, 2019.

(51) Int. Cl. A61F 2/24

(52) U.S. Cl. CPC A61F 2/2445; A61F 2220/0016; A61F 2230/0091; A61F 2250/001

(57) ABSTRACT

An anchor configured to maximize the surface area of the anchor to improve anchor retention is disclosed. The anchor may be configured with a width that differs from a thickness of the anchor. In some embodiments, the anchor be configured as a helical ribbon defining a central lumen about a central axis extending through the helical ribbon. The helical ribbon may vary in width, thickness, central lumen diameter, or a combination thereof, along its extent. The anchor may include retention features that are configured to promote tissue and/or implant interaction for improved anchor retention.

### CROSS-REFERENCE TO RELATED APPLICATION

The present application claims the benefit of priority under 35 U.S.C. § 119 to U.S. Provisional Patent Application 62/882,027, filed Aug. 2, 2019, which application is incorporated herein by reference in its entirety for all purposes.

### FIELD

The present disclosure relates generally to the field of medical devices. In particular, the present disclosure relates to medical devices, systems, and methods for annuloplasty and other cardiac treatment techniques.

### BACKGROUND

Mitral insufficiency (MI) (also referred to as mitral regurgitation or mitral incompetence) is a form of heart disease where the mitral annulus dilates excessively and the valve leaflets no longer effectively close, or coapt, during systolic contraction. Regurgitation of blood occurs during ventricular contraction and cardiac output decreases as a result.

Mitral valve annuloplasty is a procedure which seeks to reduce a dilated valve annulus to regain mitral valve competence. Surgical annuloplasty involves surgical implantation of an annuloplasty ring around a valve annulus to restore it approximately to its native configuration. Annuloplasty surgery is an invasive and time-consuming procedure that poses risks of morbidity and mortality due to stroke, thrombosis, heart attack, and extended recovery time.

Endoluminal annuloplasty is a less invasive annuloplasty method that transluminally navigates an implant to a mitral valve treatment site. Endoluminal implants may include collars, rings, expandable frames, or other structures that are affixed to the annular ring during deployment using anchors.

The efficacy of an implant may be impaired due to the chronic stress experienced by the anchors from the palpitation of the cardiac muscle. It would be desirable to maintain implant integrity by minimizing the impact of chronic stress upon implant anchors.

### SUMMARY

Embodiments of the present disclosure relate to an anchor configured to increase anchor interaction with one or both of cardiac tissue and the implant to maintain implant integrity. In accordance with some aspects of the present disclosure, the anchor shaft is configured to present an element that increases surface area for contact with tissue in which the anchor shaft is inserted, such that the anchor shaft presents a surface area greater than the surface area presented by prior art anchor shafts, to the tissue to be engaged by the anchor shaft. In some embodiments, the anchor shaft is in a helical configuration to form a helical anchor. The increase in surface area presented by the anchor shaft for engagement with tissue may be achieved in a variety of manners as disclosed herewith. In some embodiments, the cross-sectional shape of the shaft is modified to present a surface with a greater surface area for contact with tissue than previously provided. In some embodiments, the cross-sectional shape of the shaft has more than one side or surface contacting the tissue. In some embodiments, the cross-sectional shape is canted or angled with respect to the longitudinal axis or central axis or translation axis of the anchor to provide more than one side or surface for engagement with tissue.

In various embodiments, one or both of the width or the thickness of the anchor shaft may vary over the length of the anchor shaft. The width or the thickness of the anchor shaft may reduce at least once towards the distal tip of the anchor shaft.

A central lumen having a length along the central axis and defined by the anchor shaft may vary in diameter along the length of the central lumen. The diameter of the central lumen may reduce at least once towards the distal tip of the anchor shaft.

The anchor may further (or alternatively) include a retention feature disposed on the anchor shaft. The width of the anchor shaft may be angularly oriented relative to the central axis. At least one opening may extend through the width of the anchor shaft. The retention feature may include one or more barbs disposed on the anchor shaft. The retention feature may be configured to retain the anchor within an anchor housing of an implant. The width of the retention feature may be oriented parallel to the central axis, and the at least one opening may extend perpendicularly to central axis through the width of the anchor shaft. The at least one opening may be one of a plurality of openings disposed on the anchor shaft, each opening disposed on or through a surface of the anchor shaft, or both. The anchor may include at least one filler disposed in the at least one opening, the at least one filler including a drug, an extracellular matrix, a fibrous matrix, a mesh, a braid, or some combination thereof. The anchor may be formed of a laser cut hypo tube.

According to another aspect an implant includes a frame configured for a valve annulus and a plurality of anchors, coupled to the frame. Each anchor may include a proximal head, a distal tip, and an anchor shaft disposed between the proximal head and the distal tip, the anchor shaft having a width and a thickness. In some embodiments, the thickness is different from the width. In some embodiments, the anchor shaft presents more than one surface for engagement with the tissue. The anchor shaft may be helically disposed about a central axis extending from the proximal head to the distal tip of the anchor and include a retention feature configured to secure the anchor shaft to one or both of the frame and the valve annulus.

In various embodiments, one or both of the width and the thickness of the anchor shaft may vary at least once over a length of the anchor shaft. The retention feature may include a surface texture of the anchor shaft, a protuberance on the anchor shaft, or an opening on or through the anchor shaft, or some combination thereof. The frame may include an expandable frame having adjacent struts joined at a distal end of the adjacent struts, the adjacent struts configured to support at least one anchor, and where the retention feature of the at least one anchor retains the at least one anchor within the distal end of the adjacent struts.

According to another aspect a method of annuloplasty includes the steps of positioning an implant proximate a valve annulus. The implant may include a frame including a plurality of struts joined at an apex, an anchor housing disposed on the apex, and an anchor supported by the anchor housing. The anchor may include an anchor shaft having a width and a thickness, the thickness different from the width, and at least one backout feature configured to inhibit proximal translation of the anchor shaft through the anchor housing. The method may include the steps of driving the anchor through the anchor housing of the implant into annular tissue to expose the at least one backout feature to tissue and releasing the anchor, where the at least one backout feature interacts with at least one of the implant and the tissue to inhibit proximal translation of the anchor shaft through the anchor housing.

In one embodiment, the at least one backout feature may include a surface texture of the anchor shaft, a protuberance on the anchor shaft, or an opening on or through the anchor shaft, or some combination thereof. In one embodiment, one or both of the width and the thickness of the anchor shaft varies at least once over a length of the anchor shaft.

### BRIEF DESCRIPTION OF THE DRAWINGS

Non-limiting embodiments of the present disclosure are described by way of examples with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1C illustrate anchors with cross-sections configured according to various embodiments of the present disclosure;

FIGS. 2A-2F illustrate anchor shafts configured according to various embodiments of the present disclosure;

FIG. 3 illustrates an exemplary embodiment of an anchor as disclosed herein;

FIGS. 4A and 4B illustrate embodiments of anchors with retention features as disclosed herein;

FIG. 5 illustrates an exemplary embodiment of an anchor with retention features as disclosed herein;

FIGS. 6A-6D illustrate examples of retention features that may be used with anchors disclosed herein;

FIGS. 7A-7C illustrate various embodiments of anchors comprising retention features as disclosed herein;

FIGS. 8A-8C illustrate embodiments of various implants that may benefit from the use of anchors configured as disclosed herein; and

FIG. 9 illustrates a control handle that may be used to control implants such as those disclosed in FIGS. 8A-8C to deploy anchors as disclosed herein to a valve treatment site.

### DETAILED DESCRIPTION

Stresses and strains resulting from the palpatory motion of the heart may adversely affect the integrity of implant anchoring over time. For example, anchor affixation may degrade, and the anchor may become partially or fully released from its original anchoring location. Such anchor backout may impair the efficacy of the implant. To overcome these and other issues, an anchor as disclosed herein is configured to maximize the contact surface area between an anchor and exposed tissue to improve integration between the tissue and the anchor. Accordingly, in various embodiments, the anchor may be configured such that a width differs from a thickness of the anchor. In some embodiments the anchor may be generally ribbon shaped. In various embodiments, the ribbon may be helically shaped, may vary in width and/or thickness along its extent, may include holes, features or textures that aid in tissue integration, or a combination of any one or more such attributes.

For example, in some embodiments, the anchor may include retention features that are configured to promote tissue interaction and/or ingrowth with the anchor to improve tissue/anchor interaction and as a result anchor retention. In some embodiments, the retention features may also improve retention of the anchor within the implant in the presence of chronic palpatory forces.

These and other beneficial aspects of the disclosed anchor are now described below. It should be noted that although embodiments of the present disclosure may be described with specific reference to mitral valves, the principles disclosed herein may be readily adapted to facilitate reconstruction of any valve annulus, for example including a tricuspid valve annulus and/or may similarly benefit any other dilatation, valve incompetency, valve leakage and other similar heart failure conditions.

As used herein, the term “distal” refers to the end farthest away from the medical professional when introducing a medical device into a patient, while the term “proximal” refers to the end closest to the medical professional when introducing a medical device into a patient.

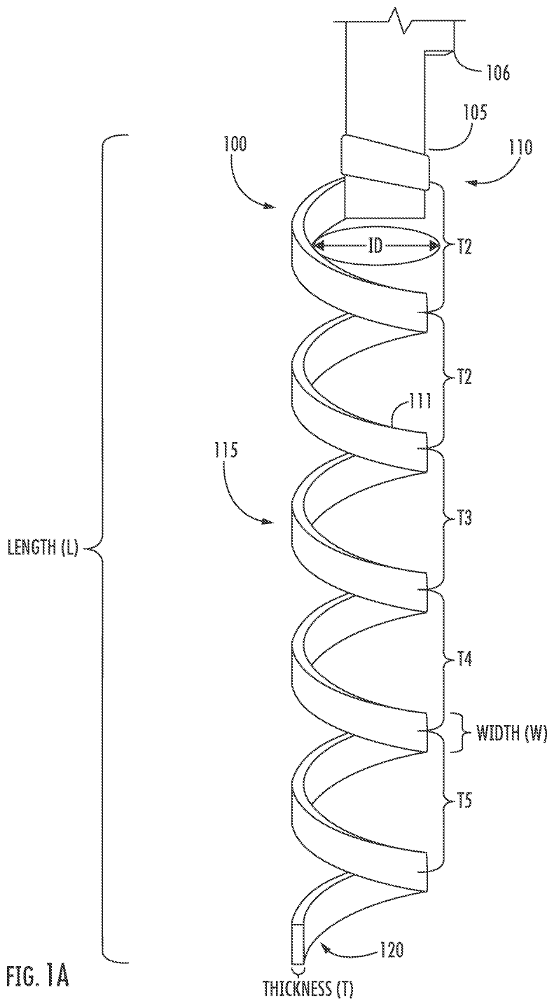

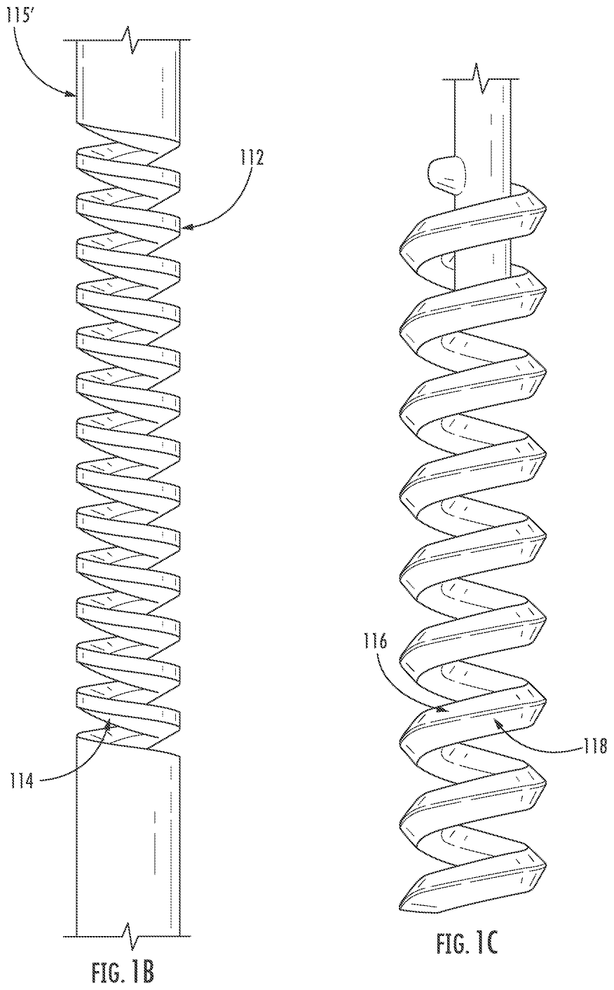

FIG. 1 illustrates an exemplary embodiment of an anchor 100 configured for cardiac use. The anchor 100 is shown to comprise a proximal end 110, a distal tip 120 and an anchor shaft 115 extending therebetween. An anchor head 105 is disposed on the proximal end 110 of the anchor 100. The anchor head 105 may include a drive coupler 106 for engaging the anchor 100 with a driver configured to rotate the anchor to axially translate the anchor and drive the anchor into tissue. For example, the drive coupler 106 may be a feature that cooperates with a complementary feature of the driver to transmit force from the driver to the drive coupler to translate the anchor. While a particular drive coupler 106 is shown, the present disclosure is not limited to the use of any particular form of drive coupler.

The anchor shaft 115 is coupled to the anchor head 105. In some embodiments, the anchor head may be bonded to or integral with the anchor shaft 115. The anchor shaft 115 comprises a length L, a width W, and a thickness T. In contrast to prior art anchors, many of which are formed from a rounded wire, the anchor shaft 115 is advantageously configured to maximize contact surface area with tissue, and thus comprises a width W that exceeds the thickness T of the shaft 115. The resulting ‘ribbon’ shaped anchor 100 is provided. In various embodiments, the relatively wider width of a ribbon surface is oriented for maximal tissue contact. By maximizing tissue contact, the opportunity for integration between the anchor 100 and surrounding tissue is improved, and anchor migration and backout may be reduced.

In various embodiments, the surface area of the anchor contacting the tissue may be increased by other modifications to the cross-sectional shape of the turns of the anchor. For instance, the anchor shaft may have a shape (with three or more sides) with more than one side or surface thereof presented for engagement with tissue. For instance, in an anchor with a quadrilateral shape or cross-section, each turn of the helical tissue anchor may be canted to present two sides of the quadrilateral shape to the tissue. Such canted configuration or effect may be formed by a laser cutting process applied to the material, e.g., tube or shaft, from which the anchor is formed. An example of an anchor formed by a laser cutting process is shown in FIG. 1B, presenting, in addition to the outer surface 112 of the anchor (typically extending along the longitudinal axis or translation axis of the anchor), an additional canted surface 114 for increasing the contact area of the anchor shaft 115′ with the tissue. If a wire having a quadrilateral cross-section is used to form the helical tissue anchor, the anchor may have a canted wire configuration with the wire being canted or angled to present two walls of its quadrilateral cross-section to tissue to be contacted by the anchor/wire. Such configuration can be achieved, for example, by winding a square (or other quadrilateral-cross-sectioned) wire at an angle to form the helical coil of the anchor, or by twisting the square (or other quadrilateral-cross-sectioned) wire during formation into a helical/coiled anchor. An example of a wire with a quadrilateral cross-section formed into an anchor shaft 115″ with a canted surface for contacting tissue (such that two walls 116, 118 of the quadrilateral surface of the wire are positioned to contact tissue) is illustrated in FIG. 1C.

According to one aspect, an anchor such as that disclosed herein for use in a cardiac cavity may be formed from a suitable biocompatible metal alloy such as stainless steel, cobalt chromium, platinum iridium, nickel titanium, other suitable materials, or combinations thereof. The distal end 120 of the shaft 115 may be sharpened at its distal point, or leading turn, so as to facilitate penetration into the cardiac tissue. Each anchor 100 may be from about ten to about fifteen millimeters (mm) in total axial length. In some embodiments, the anchors 100 may be shorter or longer than ten to fifteen millimeters (mm) in total axial length. By “total” axial length it is meant the axial length of the anchor 100 from the end of the distal penetrating tip 120 to the opposite, proximal end of the head 105. The Length L of the shaft 115 may be from about six to about twelve millimeters (mm). In some embodiments, the shaft 115 may be shorter or longer than six to twelve millimeters (mm) in axial length. The anchor head 105 and/or other non-helical portions of the anchor 100 may be from about three to about four millimeters (mm) in axial length. In some embodiments, the anchor head 105 and/or other non-helical portions may be shorter or longer than three to four millimeters (mm) in axial length.

In some embodiments, the shaft 115 may be laser cut from a stainless steel hypo tube formed of full hard temper, type 304 stainless steel, resulting in an anchor having a central lumen extending therethrough defined by the turns of the helical anchor. In other embodiments, the anchor 100 may be cut from a stainless steel sheet and shaped to obtain the helical anchor configuration. In some embodiments, the thickness T of the stainless steel sheet and/or hypo tube may range from between 0.020 mm to about 2 mm In some embodiments, the thickness T may increase, decrease and/or otherwise vary along the length L of the anchor shaft 110. In some embodiments, the inner diameter (ID) of the central lumen may range from between 1 mm to about 3 mm. In some embodiments, the inner diameter (ID) may increase, decrease or otherwise vary along the length L of the anchor shaft 115.

In some embodiments, the width W of the anchor may range from between 0.5 mm to about 2 mm. In some embodiments, the width W may increase, decrease or otherwise vary along the length L of the anchor shaft 115. In general, the width is selected to maximize the surface area contact between the anchor and neighboring tissue to secure the anchor to the tissue. Although the anchor shaft 115 is shown to include five turns (T1-T5) (“pitch”), in various embodiments, the number of turns of the anchor may be from about ten per inch to about thirty-six per inch. In some embodiments the distal tip 120 as well as at least a portion of edges 111 of the anchor 110 are sharpened and/or beveled to facilitate tissue engagement.

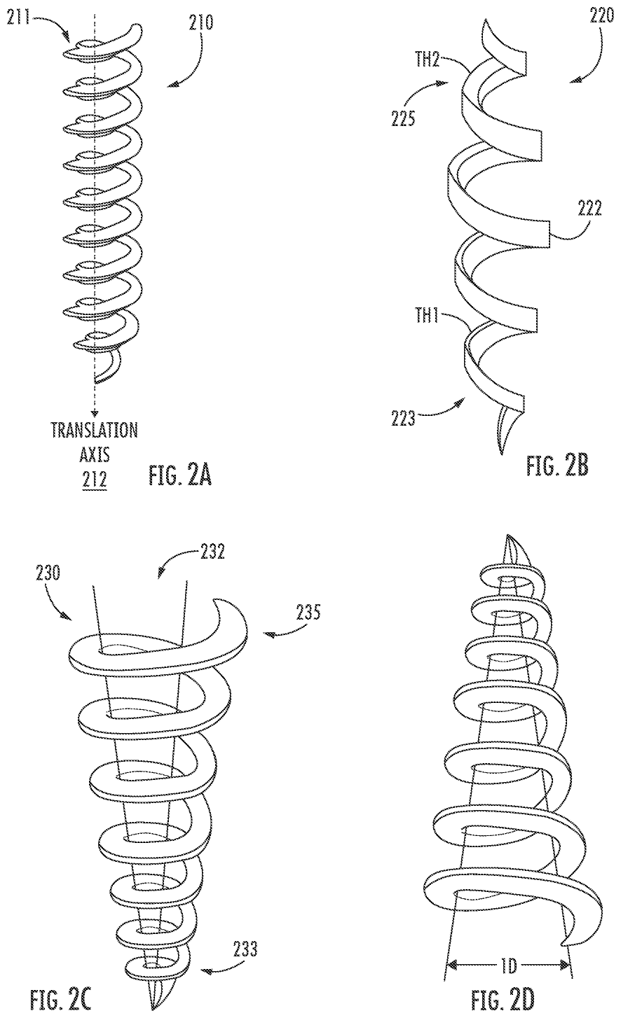

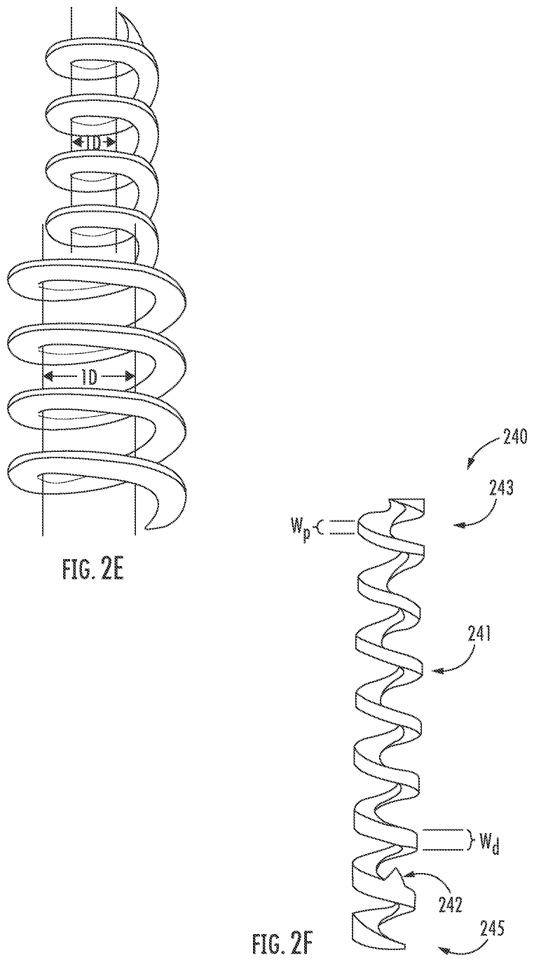

FIGS. 2A-2F illustrate various other embodiments of an anchor shaft that may be part of an anchor as disclosed herein. In the embodiment of FIG. 2A, the surface 211 of an anchor shaft 210 having a relatively larger width compared to its thickness is shown oriented in a plane perpendicular to a translation axis 212 of the anchor. For the purposes of this disclosure, that portion of the anchor shaft having the relatively larger width is referred to as the width surface. In FIG. 2A the anchor shaft is configured such that the width surface 211 is oriented relatively perpendicular to the translation axis 212 of the anchor shaft 210. Orienting the width surface 211 of the ribbon perpendicular to the translation axis 212 increases resistance to anchor backout. While the width surface is shown oriented relatively perpendicular to the translation axis 212 in FIG. 2A, the present disclosure is not limited to any particular angular orientation of the width surface 211 relative to the translation axis 212, and embodiments having different angular orientations ranging from 0-180 degrees are within the scope of this disclosure.

FIG. 2B illustrates another embodiment of an anchor shaft 220 as disclosed herein, wherein anchor shaft 220 varies in both central lumen diameter and thickness over the extent of the anchor shaft 220. In FIG. 2B, the ribbon surface 222 is oriented to engage tissue surrounding the central lumen of the anchor shaft. Such an arrangement reduces the potential for pull out by advantageously increasing the volume and extent of anchored tissue. Also shown in FIG. 2B, the thickness of the anchor shaft 220 varies from TH1 towards a distal end 223, increasing to TH2 towards a proximal end 225. An anchor shaft that increases in thickness as it extends proximally may facilitate introduction of the anchor into tissue while providing proximal anchor strength against chronic forces of a tissue surface.

FIGS. 2C-2E illustrate another embodiment of an anchor shaft 230, wherein the diameter (inner diameter ID) of the central lumen 232 defined by the anchor shaft 230 tapers from a relatively larger diameter at a proximal end 235 of the anchor shaft 230 to a relatively smaller diameter at a distal end 233. The diameter (inner diameter ID) of the central lumen also may taper from a relatively smaller diameter at the proximal end of the anchor to a relatively larger diameter at the distal end, as illustrated in FIG. 2D. The transition from large to small or small to large may happen in a stepwise manner instead of a taper, as illustrated in FIG. 2E. In addition, as shown in FIG. 2C, the pitch (number of turns per mm) of the anchor shaft 230 is shown to vary along the extent of the anchor shaft 230, for example increasing towards the distal end 233, and decreasing towards the proximal end 235. Such an anchor may advantageously increase the involvement of tissue in the anchoring process and more effectively distribute forces at a tissue anchoring surface. It will be appreciated that such variations in pitch may apply to any or all of the embodiments disclosed herein.

FIG. 2F illustrates another embodiment of an anchor shaft 240 wherein a width differs between a proximal end 243 and a distal end 245, such that a width Wₚ of the proximal end 243 of the anchor shaft 240 is narrower than the width Wd of the distal end 245 of the anchor shaft 240. Such an arrangement may provide increased resiliency to chronic forces on the proximal end, while providing structural integrity to increase anchoring strength at the distal end. As described in more detail below, a retention feature 242 may be disposed near distal end 245 to improve tissue integration with the anchor shaft 240.

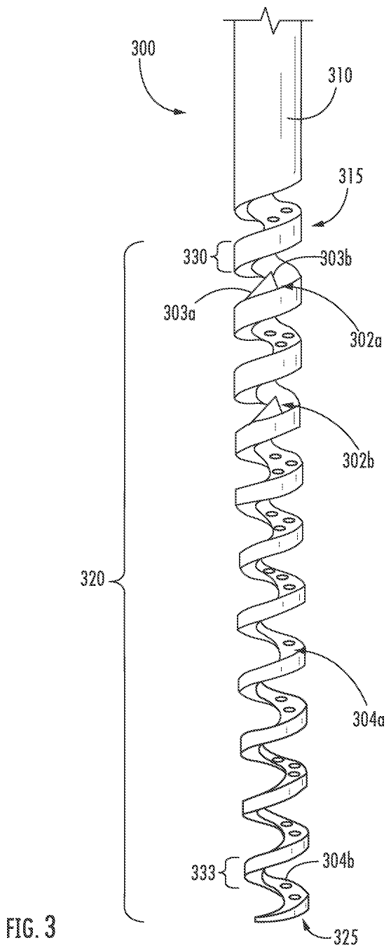

FIG. 3 illustrates another embodiment of an anchor 300 having a proximal end 315, a distal tip 325 and a shaft 320 extending therebetween. A drive tube 310 is shown coupled to an anchor head (not shown) of the anchor 300, wherein the drive tube 310 may provide torque to rotate or otherwise drive the anchor 300 into tissue.

The anchor 300 is shown constructed of a ribbon like material that varies in thickness and/or width as the shaft extends distally. Thus, turn 330, which is more proximally positioned, includes a wider surface area than turn 333. Such an arrangement may facilitate the initial driving of the anchor into tissue, which reduces the opportunity for anchor backout.

According to various embodiments, the anchors may be configured with one or more retention features. The retention features may be configured for similar or different purposes. For example, the retention features may be configured to increase interaction between the anchor and the tissue to aid securing of the anchor to the treatment site. The retention features may further be configured to decrease the potential that the anchor may be released from the implant. FIGS. 3-7C illustrate various retention features. Each retention feature may be used alone or in combination with other disclosed retention features. The retention features may be included at one location on the anchor or at multiple locations on the anchor. The retention features may be evenly distributed over the anchor, or alternatively may be unevenly distributed over the anchor. Retention features that serve similar purposes are considered equivalents to and within the scope of this disclosure.

The anchor 300 of FIG. 3 is shown to include two forms of retention features including a pair of barbs 302a and 302b and a plurality of holes such as holes 304a and 304b. In one embodiment, the barbs 302a, 302b include protuberances that are disposed along on the anchor 300, such as on an edge or a surface. The protuberances increase the surface area of the anchor and concomitantly the interaction between the anchor and the tissue. In one embodiment, the barbs may be advantageously shaped to assist distal translation and resist proximal translation of the anchor. For example, barb 302a comprises an angular barb having a sloping edge 303a that enables the barb to ride within a cut made by the anchor during distal translation and an apex 303b that acts against tissue during proximal withdrawal of the anchor to resist back out.

Holes such as hole 304a and 304b may be holes that are at least partially cut into and/or through the ribbon surface. In some embodiments, the holes may be shaped and/or sized to promote tissue ingrowth. For example, the holes may comprise pores with a diameter of at least about 20 μm, or at least about 50 μm, or at least about 75 μm, and a diameter of at most about 1000 μm, or at most about 750 μm, or at most about 500 μm. For example, pores with a diameter in the range of 700 μm+/1 15%, or 100-500 μm, or 100-250 μm may be used. The pores may extend through the ribbon anchor or partially through the ribbon anchor. In some embodiments, one or more of the pores and/or holes may be filled or partially filled with a filler such as a drug, a fibrous matrix, an extracellular matrix (ECM), a mesh, a braid, or other mechanism, or combination thereof to promote tissue ingrowth. In various embodiments, the numbers of holes/pores may vary along the extent of the anchor shaft 320. In some embodiments, the number of pores may vary based on the pitch and location of a particular point on the anchor, for example, as shown in FIG. 3, it may be advantageous to provide more holes towards a proximal end of an anchor, where the anchor is subject to greater pull out forces.

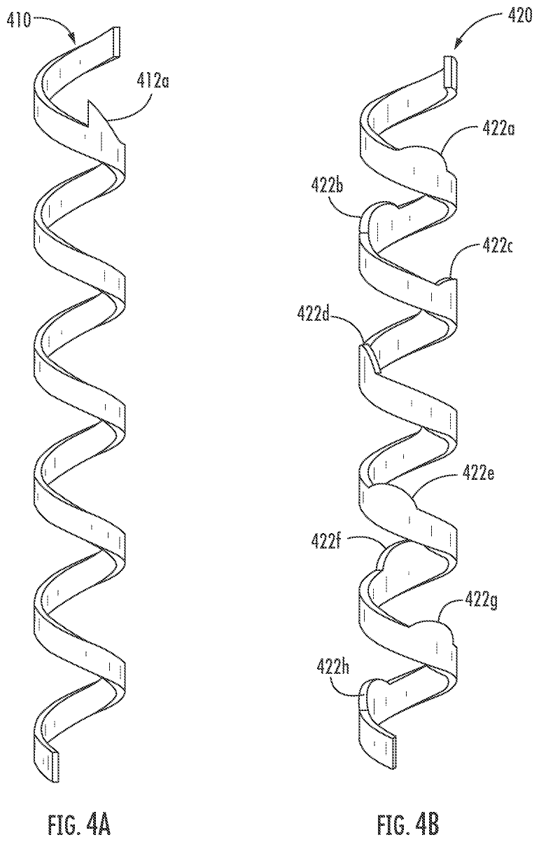

FIGS. 4A and 4B illustrate two embodiments of anchor shafts configured according to the principles disclosed herein. Anchor shaft 410 of FIG. 4A is shown to include a single barb 412a, disposed on a proximal edge of a turn of anchor shaft 410. As in FIG. 3, the shape of barb 412a facilitates distal translation, but resists against proximal translation of the anchor shaft 410. In some embodiments it may be determined that a single barb placed at a proximal edge may sufficiently counteract the backout forces experienced closer to the surface of the annulus.

FIG. 4B illustrates another embodiment of an anchor shaft 420, shown including a plurality of protuberances or nubs 422a-422h disposed at regular intervals along the anchor shaft 420. The protuberances increase the overall surface area interaction between the anchor shaft 420 and surrounding tissue. In some embodiments, the protuberances may include beveled or sharpened edges, configured to scar surrounding tissue to increase engagement with the anchor. In some embodiments the anchor shaft 420 can include any number of protuberances 422a-422h.

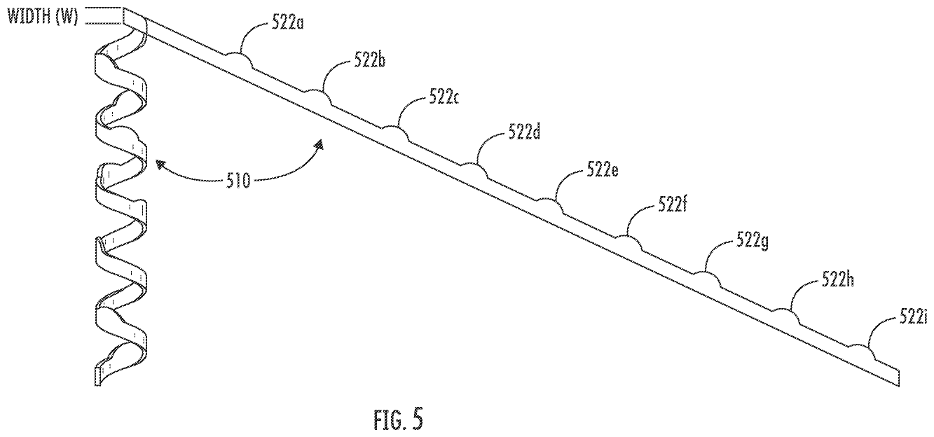

One advantage of the anchors disclosed herein is their ease of manufacturability. As mentioned previously, in one embodiment, the anchors may be laser cut from hypo tubes. Alternatively, as shown in FIG. 5 an anchor shaft 510 having a width W may be cut from a stainless steel sheet and wound around a form to achieve the desired structure. Although anchor shaft 510 includes nubs 522a-522i cut into shaft 510 at regular intervals, it is appreciated that various different types of features may be cut into the anchor shaft at different and/or irregular intervals.

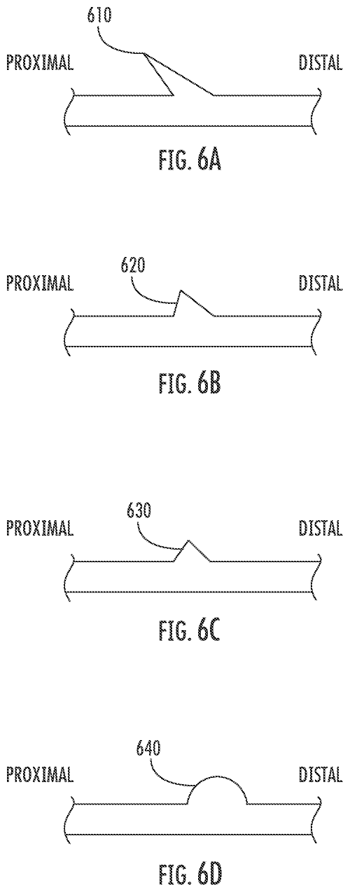

FIGS. 6A-6D illustrate examples of retention features that may be included on anchor shafts disclosed herein. Each of the features 610, 620, 630 and 640 shown in respective FIGS. 6A-6D introduce a different degree of trauma during delivery and/or may introduce different degrees of resistance to backout. The features are described with regard to proximal and distal orientations of the anchor, although it is appreciated that the features may be differently oriented. FIG. 6A illustrates an acute triangular barb which, as described with regard to previous figures, aggressively impacts tissue when moved proximally as a result of chronic forces. FIG. 6B and FIG. 6C illustrate respective obtuse triangular barb 620 and triangular barb 630, each of which may be moderately traumatic to adjacent tissue in response to chronic forces, for example to encourage tissue ingrowth but minimize scar tissue. FIG. 6D illustrates a relatively atraumatic nub 640, such as that described with regard to FIG. 5. It is appreciated that each feature may serve a different purpose. For example, barb 610 may advantageously be positioned at a location on the anchor shaft to counteract chronic forces experienced at that location. Advancing distally down the shaft, it may be more desirable to provide features geared more towards retaining the anchor in the implant and/or promoting ingrowth, such as features 620, 630 and 640. It may be advantageous to dispose the barbs such as barb 610 of FIG. 6A on the proximal end of an anchor shaft such that the sharp tip of barb 610 may impede the backout of the anchor shaft from the implant. For similar reasons, a barb such as barb 610 may be provided towards the distal end of an anchor shaft and/or intermediate to the proximal end and distal end of the anchor shaft.

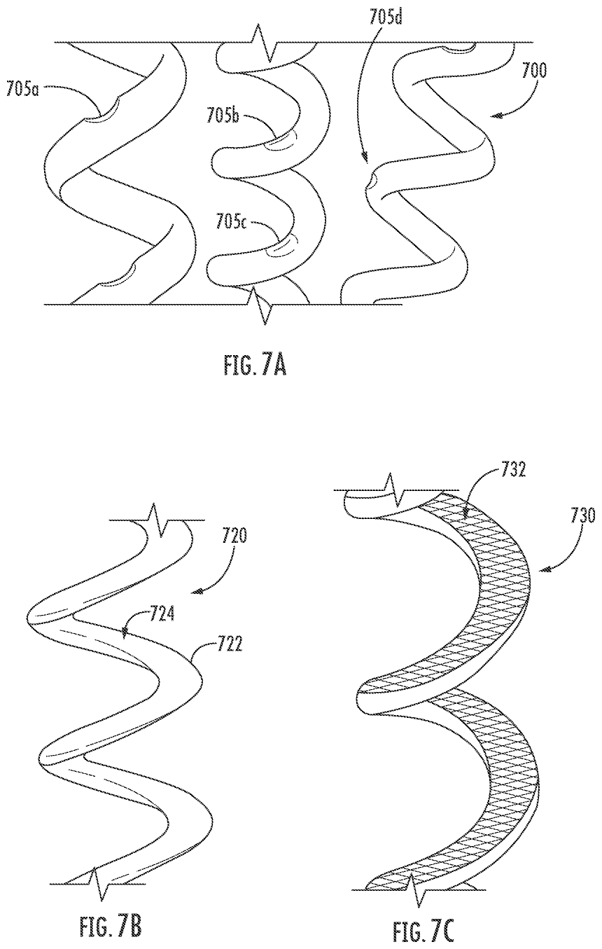

FIGS. 7A-7C illustrate various alternative embodiments of anchors including retention features disposed on anchor shafts. For example, FIG. 7A illustrates a group of anchor shafts, such as anchor shaft 700. The anchor shafts 700 are shown to include pores or divots 705a-705d disposed on a surface of the anchor shafts 700. The divots 705a-705d serve dual purposes of promoting tissue adherence to the anchors as well as providing a hard edge on the surface of the anchor shaft 700 that may interact with the implant to inhibit release of the anchor from the implant.

It should be noted that although the anchors disclosed herein have been described as generally ribbon shaped, the disclosure is not so limited. For example, FIG. 7B illustrates a helical anchor 720 comprised of a shaft 722 that is generally ovoid in shape and helically wound. In various embodiments, the ovoid shaped anchor of 720 may be disposed so that a side 724 of the shaft exposing the maximum surface area of the anchor is oriented perpendicularly to an axis of translation of the anchor 720 to increase the pull out forces and reduce the potential for back out.

FIG. 7C illustrates another embodiment of an anchor 730, comprising a texture 732 disposed on at least a portion of the surface of the anchor 730. The texture 732 may be added by coating the anchor with a material, by etching the anchor, by stamping a pattern on the anchor, or through other similar means. For example, in some embodiments, a metal powder deposition may be laser sintered on the anchor 730 using a three-dimensional printing process to provide a rough, micro porous anchor surface. Such additive surface roughness may enhance tissue ingrowth and implant endothelization. In some embodiments, a roughness of proximately 350 R a-μ inch or R a-μm 8.75 may be used, although the disclosure is not so limited.

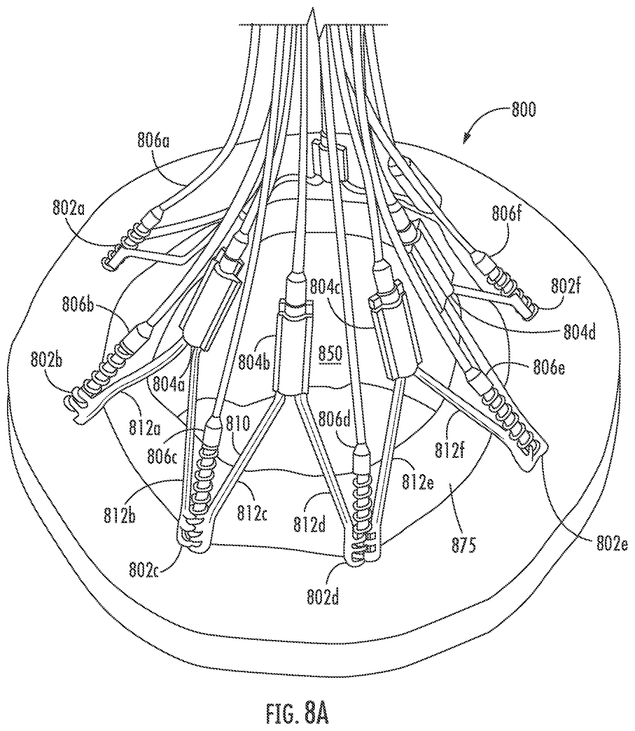

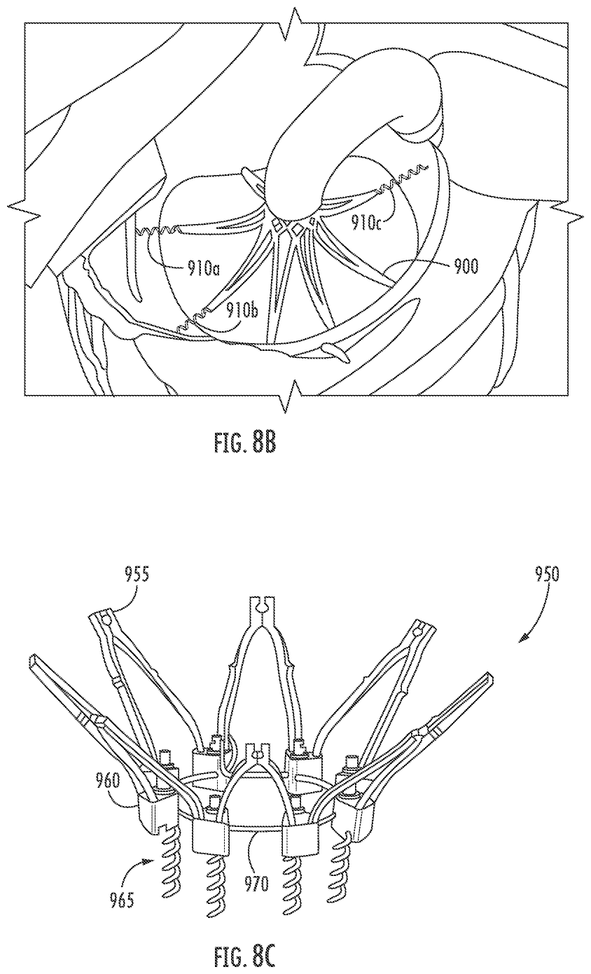

Accordingly, an improved anchor design having increased surface area to improve tissue integration has been shown and described. In various embodiments the anchors may be configured to vary in one or more of width, thickness, pitch and diameter along the extent of the anchor. The anchors may be configured with retention features configured to improve tissue adherence as well as to retain the anchors in the implant in the event of backout. It will be appreciated that the anchors may be used in a variety of cardiac implant devices, including but not limited to annular rings which are anchored into location and/or those disclosed in FIGS. 8A-8C.

FIG. 8A illustrates one embodiment of an implant which may benefit from the use of anchors disclosed herein. In FIG. 8A implant 800 has been deployed around a cardiac valve 850 in an atrium such that a plurality of anchors 802a-802f of the implant are positioned for engagement with a cardiac valve annulus 875. The implant 800 includes a generally tubular frame 810 formed from a plurality of struts (such as struts 812a-812f) joined at a proximal end by the sleeves such as sleeves 804a-804c, and at distal ends by anchors 802a-802f. Anchors 802a-802f may each be coupled to anchor drivers 806a-806f. In one embodiment, anchor drivers are configured to rotate anchors 802a-802f to drive the anchors into the tissue of the valve annulus 875 during an anchoring step of implant 800 deployment. In one embodiment, the anchors 802a-802f comprise anchors such as those disclosed herein.

FIG. 8B illustrates another embodiment of an implant that may benefit from the use of the anchors disclosed herein. The implant is shown to include a frame 900 through which a plurality of drivers may be forwarded to drive anchors 910a, 910b, and 910c into annular tissue, wherein the anchors may comprise anchors such as those disclosed in FIGS. 1-7C. FIG. 8C illustrates a third embodiment of an implant 950 that may benefit from the use of anchors disclosed herein. The implant 950 is shown to include a frame 955 comprising a plurality of struts joined at distal ends by anchor housings 960. Each anchor housing 960 is configured to translatably support an anchor such as anchor 965, which may be any of the anchors disclosed in FIGS. 1-7C. A lasso 970 may be coupled to the anchor housings, where the lasso may be used to pull together anchor housings and thus anchors, to reduce the size of a valve annulus. According to one aspect, the retention features disclosed herein, including the barbs, nubs, textures, divots and other features may interact with the anchor housing to secure the anchor to the implant in the presence of anchor backout.

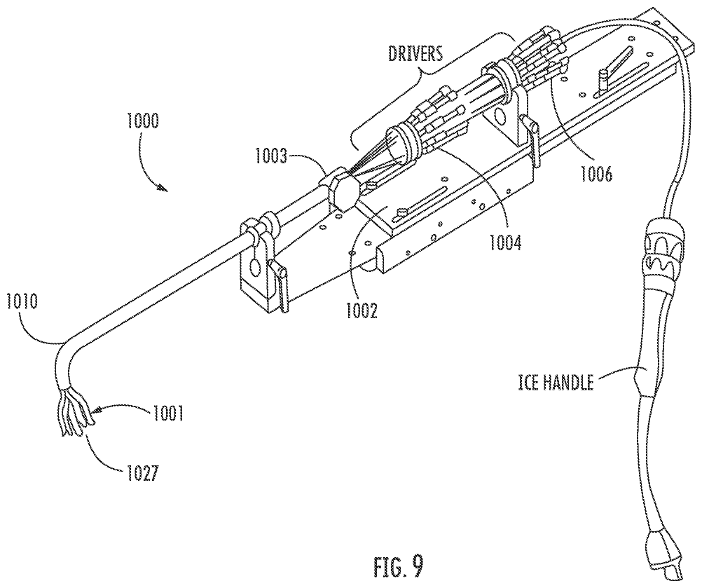

FIG. 9 is a perspective view of an exemplary deployment system 1000 that may be used to deploy an implant 1001 for annular reshaping by driving anchors such as those disclosed herein into tissue. The deployment system 1000 comprises a steerable sheath 1010, a sheath steering knob 1003, anchor knobs 1004, cinch knobs 1006, implant 1001, an Intra-Cardiac Echocardiography (ICE) probe 1027, all supported and secured to a base 1002. The cinch knobs 1006 and anchor knobs 1004 may be spring loaded to maintain tension. Rotation of the anchor knobs 1004 may rotationally advance the anchors disclosed herein into the annular tissue.

Thus, an improved anchor design having increased surface area to improve tissue integration has been shown and described. Although embodiments of the present disclosure may be described with specific reference to medical devices and systems (e.g., transluminal devices inserted through a femoral vein or the like) for selective access to heart tissue, it should be appreciated that such medical devices and systems may be used in a variety of medical procedures that require anchoring to heart tissue. The disclosed medical devices and systems may also be inserted via different access points and approaches, e.g., percutaneously, endoscopically, laparoscopically, or combinations thereof.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about,” in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g. 1 to 5 includes 1, 1.5, 2, 2.75, 3, 3.80, 4, and 5).

It is noted that references in the specification to “an embodiment,” “some embodiments,” “other embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described herein, even if not explicitly shown in a particular combination, are nevertheless contemplated as being combinable or arrangeable with each other to form other additional embodiments or to complement and/or enrich the described embodiment(s), as would be understood by one of ordinary skill in the art.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope, and concept of the disclosure as defined by the appended claims.

1-15. (canceled)

16. An anchor comprising:
a proximal head;
a distal tip; and
an anchor shaft disposed between the proximal head and the distal tip, the anchor shaft having a length, a width, and a thickness, where the anchor shaft is helically disposed along the length of the anchor shaft about a central axis extending from the proximal head to the distal tip of the anchor and presents an element that increases surface area for contact with tissue in which the anchor shaft is inserted.

17. The anchor of claim 16 wherein one or both of the width or the thickness of the anchor shaft varies over the length of the anchor shaft.

18. The anchor of claim 17, wherein one or both of the width or the thickness of the anchor shaft reduces at least once towards the distal tip of the anchor shaft.

19. The anchor of claim 18, wherein a central lumen having a length along the central axis and defined by the anchor shaft varies in diameter along the length of the central lumen.

20. The anchor of claim 19, wherein the diameter of the central lumen reduces at least once towards the distal tip of the anchor shaft.

21. The anchor of claim 16, further comprising a retention feature disposed on the anchor shaft.

22. The anchor of claim 21, wherein the width of the anchor shaft is angularly oriented relative to the central axis, and the at least one opening extends through the width of the anchor shaft.

23. The anchor of claim 21, wherein the width anchor shaft is oriented parallel to the central axis, and the at least one opening extends perpendicularly to the central axis through the width of the anchor shaft

24. The anchor of claim 21, wherein the at least one opening is one of a plurality of openings disposed on the anchor shaft, each opening disposed on or through a surface of the anchor shaft, or both.

25. The anchor of claim 24, further comprising at least one filler disposed in the at least one opening, the at least one filler includes a drug, an extracellular matrix, a fibrous matrix, a mesh, a braid, or some combination thereof.

26. The anchor of claim 22, wherein the retention feature comprises one or more barbs disposed on the anchor shaft.

27. The anchor of claim 26, wherein the retention feature is configured to retain the anchor within an anchor housing of an implant.

28. The anchor of claim 16, comprised of a laser cut hypo tube.

29. The anchor of claim 16, wherein the thickness is different from the width.

30. An implant, comprising:
a frame configured for a valve annulus;
a plurality of anchors, coupled to the frame, each anchor comprising:
a proximal head;
a distal tip; and
an anchor shaft disposed between the proximal head and the distal tip, the anchor shaft having a width and a thickness, the thickness different from the width, where the anchor shaft is helically disposed about a central axis extending from the proximal head to the distal tip of the anchor and comprises a retention feature configured to secure the anchor shaft to one or both of the frame and the valve annulus.

31. The implant of claim 30, wherein one or both of the width and the thickness of the anchor shaft varies at least once over a length of the anchor shaft.

32. The implant of claim 30, wherein the retention feature includes a surface texture of the anchor shaft, a protuberance on the anchor shaft, or an opening on or through the anchor shaft, or some combination thereof.

33. The implant of claim 30, wherein the frame comprises an expandable frame having adjacent struts joined at a distal end of the adjacent struts, the adjacent struts configured to support at least one anchor, and wherein the retention feature of the at least one anchor retains the at least one anchor within the distal end of the adjacent struts.

34. A method of annuloplasty comprising:
positioning an implant proximate a valve annulus, the implant comprising a frame comprising a plurality of struts joined at an apex, an anchor housing disposed on the apex, and an anchor supported by the anchor housing, the anchor including an anchor shaft having a width and a thickness, the thickness different from the width, the anchor shaft including at least one backout feature configured to inhibit proximal translation of the anchor shaft through the anchor housing;
driving the anchor through the anchor housing of the implant into annular tissue to expose the at least one backout feature to tissue; and
releasing the anchor, wherein the at least one backout feature interacts with at least one of the implant and the tissue to inhibit proximal translation of the anchor shaft through the anchor housing.

35. The method of claim 34, wherein the at least one backout feature includes a surface texture of the anchor shaft, a protuberance on the anchor shaft, or an opening on or through the anchor shaft, or some combination thereof.
